(19) United States
(12) Patent Application Publication
(10) Pub. No.: US 2024/0209066 A1
(43) Pub. Date: Jun. 27, 2024

(54) 2019 NOVEL CORONAVIRUS ANTIBODY-CONTAINING PHARMACEUTICAL FORMULATIONS

(71) Applicant: Eli Lilly and Company, Indianapolis, IN (US)

(72) Inventor: Yu TANG, Carmel, IN (US)

(21) Appl. No.: 18/567,814

(22) PCT Filed: Jun. 8, 2022
(86) PCT No.: PCT/US2022/032586

§ 371 Date: Dec. 7, 2023

Related U.S. Application Data
(60) Provisional application No. 63/208,188, filed on Jun. 8, 2021.

(51) Int. Cl. C07K 16/10; A61K 47/22; A61K 47/26

(52) U.S. Cl. CPC C07K 16/1003; A61K 47/22; A61K 47/26

(57) ABSTRACT

A pharmaceutical formulation is provided that includes a 2019 novel coronavirus (2019-nCOV) antibody and agents that provide commercially acceptable shelf-life stability, in-use stability and acceptable patient injection site experience. Such formulations can be used for attenuating, preventing and/or treating 2019-nCoV infection or COVID-19.

### TECHNICAL FIELD

This disclosure generally relates to biology and medicine, and more specifically it relates to compositions such as pharmaceutical formulations having a human, monoclonal antibody (Ab) to severe acute respiratory syndrome coronavirus 2 (SARS-COV-2; hereinafter referred to as 2019 novel coronavirus or 2019-nCOV) having a high neutralizing activity. The formulations additionally include agents that provide commercially acceptable shelf-life stability, in-use stability and acceptable patient injection site experience. Such formulations can be used for attenuating, preventing and/or treating 2019-nCoV infection.

### BACKGROUND

First officially noticed at the end of 2019 in Wuhan, China, a pneumonia caused by 2019-nCOV rapidly spread throughout China and the world. The disease caused by 2019-CoV has been named Coronavirus Disease 2019 (COVID-19) by the World Health Organization (WHO). The impact of this COVID-19 has been unprecedented, and as of March 2021, cases have reached over 126 million cases with more than 2.8 million deaths globally, which demonstrates a continuing critical need for prophylactic and therapeutic treatments. While vaccines remain a primary option for COVID-19 prevention, neutralizing monoclonal Abs have shown to be an effective treatment option for those already infected, as well as having the potential to prevent infection in those already exposed to 2019-nCOV, which can be of particular benefit to certain high-risk sub-populations.

Intl. Patent Application Publication No. WO 2021/169932. describes a monoclonal 2019-nCOV Ab, known as etesevimab, LY-CoV016, JS016 or CB6, which can be used for attenuating, preventing and/or treating 2019-nCoV infection.

Because proteins, including Abs, are larger and more complex than traditional organic and inorganic therapeutics (e.g., possessing multiple functional groups in addition to complex three-dimensional structures), the formulation of such proteins often poses difficulties. For example, for a protein to remain biologically active, a formulation must preserve intact the conformational integrity of at least a core sequence of its amino acids while at the same time protecting its multiple functional groups from degradation. Degradation pathways for proteins can involve chemical instability (e.g., any process that involves modification of the protein by bond formation or cleavage resulting in a new chemical entity) or physical instability (e.g., changes in the higher order structure of the protein). Chemical instability can result from deamidation, racemization, hydrolysis, oxidation, beta elimination or disulfide exchange. Physical instability can result from denaturation, aggregation, precipitation or adsorption, for example. The three most common protein degradation pathways are protein aggregation, deamidation and oxidation. See, Cleland et al. (1993) Crit. Rev. Ther. Drug Carrier Syst. 10:307-377.

In view of the serious threat to global public health, there is a need for safe and effective formulations including a 2019-nCOV Ab for use as prophylactics and therapeutics, especially for attenuating, preventing and/or treating 2019-nCoV infection.

### BRIEF SUMMARY

To address this need, the disclosure describes compositions such as pharmaceutical formulations that include at least a 2019-nCOV Ab, known as etesevimab, or a pharmaceutically acceptable salt thereof. Such formulations can be used for attenuating, preventing and/or treating 2019-nCoV infection or COVID-19. In addition, such formulations can be packaged for intravenous (IV) or subcutaneous (SQ) administration as described herein with maintenance of, for example, product stability and other desirable attributes.

In particular, formulations are described that include a 2019-nCOV Ab, a histidine buffer, a polyol or tonicity agent, and a surfactant.

In some instances, the 2019-nCOV Ab includes heavy chain complementarity-determining regions (HCDR) HCDR1, HCDR2 and HCDR3 and light chain complementarity-determining regions (LCDR) LCDR1, LCDR2 and LCDR3, where HCDR1 has the amino acid sequence of SEQ ID NO:1, HCDR2 has the amino acid sequence of SEQ ID NO:2 and HCDR3 has the amino acid sequence of SEQ ID NO:3, and where LCDR1 has the amino acid sequence of SEQ ID NO:4, LCDR2 has the amino acid sequence of SEQ ID NO:5 and LCDR3 has the amino acid sequence of SEQ ID NO:6.

In other instances, the 2019-nCOV Ab includes a heavy chain variable region (HCVR), where the HCVR has the amino acid sequence of SEQ ID NO:7. Alternatively, the 2019-nCOVAb includes a light chain variable region (LCVR), where the LCVR has the amino acid sequence of SEQ ID NO:8. Alternatively still, the 2019-nCOV Ab includes a HCVR having the amino acid sequence of SEQ ID NO:7 and a LCVR having the amino acid sequence of SEQ ID NO:8.

In yet other instances, the 2019-nCOV Ab includes a heavy chain (HC), where the HC has the amino acid sequence of SEQ ID NO:9. Alternatively, the 2019-nCOV Ab includes a light chain (LC), where the LC has the amino acid sequence of SEQ ID NO:10. Alternatively still, the 2019-nCOV Ab includes a HC having the amino acid sequence of SEQ ID NO:9 and a LC having the amino acid sequence of SEQ ID NO: 10.

In any of the above, the 2019-nCOV Ab can be at a concentration from about 25 mg/mL to about 150 mg/mL. In some instances, the 2019-nCOV Ab is at a concentration of 25 mg/mL, 30 mg/mL, 35 mg/mL, 40 mg/mL, 45 mg/mL or 50 mg/mL. In certain instances, the 2019-nCOV Ab is at a concentration of 30 mg/mL to 40 mg/mL, especially 35 mg/mL.

In some instances, the histidine buffer can be L-histidine and/or L-histidine hydrochloride monohydrate at a pH from about 5.5 to about 7.0.

In any of the above, the histidine buffer can be at a concentration from about 5 mM to about 40 mM. In certain instances, the histidine buffer is at a concentration of 20 mM at a pH of 6.0.

In some instances, the polyol or tonicity agent can be mannitol or sucrose. In certain instances, the polyol or tonicity agent is sucrose.

In any of the above, the polyol or tonicity agent can be at a concentration from about 1% (w/v) to about 10% (w/v). In some instances, the polyol or tonicity agent can be at a concentration of about 4.5% (w/v) to about 8.5% (w/v). In certain instances, the polyol or tonicity agent is at a concentration of about 8.0% (w/v). In particular instances, the polyol or tonicity agent is sucrose at a concentration of 8.04% (w/v).

In some instances, the surfactant can be a polysorbate. In certain instances, the surfactant is polysorbate 80.

In any of the above, the surfactant can be at a concentration of about 0.01% (w/v) to about 0.1% (w/v). In certain instances, the surfactant is at a concentration of about 0.02% (w/v) to about 0.05% (w/v). In particular instances, the surfactant is polysorbate 80 at a concentration of 0.05% (w/v).

In one instance, the formulation includes a 2019-nCOV Ab having a HCDR1 having the amino acid sequence of SEQ ID NO:1, a HCDR2 having the amino acid sequence of SEQ ID NO:2, a HCDR3 having the amino acid sequence of SEQ ID NO:3, a LCDR1 having the amino acid sequence of SEQ ID NO:4, a LCDR2 having the amino acid sequence of SEQ ID NO:5 and a LCDR3 having the amino acid sequence of SEQ ID NO:6 in a L-histidine buffer at a concentration of about 20 mM at pH 6.0, sucrose at a concentration of about 8.04% (w/v) and polysorbate 80 at a concentration of about 0.05% (w/v).

In another instance, the formulation includes a 2019-nCOV Ab having a HCVR having the amino acid sequence of SEQ ID NO:7 and a LCVR having the amino acid sequence of SEQ ID NO:8 in a L-histidine buffer at a concentration of about 20 mM at pH 6.0, sucrose at a concentration of about 8.04% (w/v) and polysorbate 80 at a concentration of about 0.05% (w/v).

In yet another instances, the formulation includes a 2019-nCOV Ab having a HC having the amino acid sequence of SEQ ID NO:9 and a LC having the amino acid sequence of SEQ ID NO:10 in a L-histidine buffer at a concentration of about 20 mM at pH 6.0, sucrose at a concentration of about 8.04% (w/v) and polysorbate 80 at a concentration of about 0.05% (w/v).

In some instances, the formulation can include one or more additional Abs to 2019-nCOV.

The disclosure also describes methods of attenuating, preventing and/or treating 2019-nCOV infection or COVID-19, where such methods include at least a step of administering to an individual in need an effective amount/dose of a formulation herein. In some instances, the composition is administered about once weekly. In some instances, the individual has a confirmed 2019-nCoV infection. In other instances, the individual is at risk for exposure to 2019-nCOV. In other instances, the individual is recently exposed to 2019-nCoV infection (i.e., but not yet having a confirmed 2019-nCoV infection).

The disclosure further describes a composition herein for use as a medicament.

The disclosure further describes a composition herein for use in the treatment of 2019-nCoV infection.

The disclosure further describes an article of manufacture including a formulation herein. In some instances, the article of manufacture is a multi-use vial. In some instances, the article of manufacture is a pre-filled syringe. In some instances, the article of manufacture is an automatic injection apparatus (“auto-injector”; see, e.g., U.S. Pat. No. 8,734,394). In some instances, the article of manufacture is a pump for continuous perfusion, especially a pump for subcutaneous infusion.

### DETAILED DESCRIPTION

Unless defined otherwise, all technical and scientific terms used herein have the same meaning as commonly understood by one of skill in the art to which the disclosure pertains. Although any methods and materials similar to or equivalent to those described herein can be used in the practice or testing of the incretin analogs, pharmaceutical compositions and methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

### Definitions

As used herein, “about” means within a statistically meaningful range of a value or values such as, for example, a stated concentration, length, molecular weight, pH, sequence identity, time frame, temperature or volume. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, “affinity” means a strength of a 2019-nCOV Ab's binding to an epitope on 2019-nCOV, such as, for example, 2019-nCOV receptor binding domain (RBD) of the spike (S) protein (SEQ ID NO: 11).

As used herein, “antibody” or “Ab” and the like means a full-length Ab including two HCs and two LCs having inter- and intra-chain disulfide bonds. The amino-terminal portion of each of the four polypeptide chains includes a variable region primarily responsible for antigen recognition. Each HC includes an N-terminal HCVR and an HC constant region (HCCR). Each light chain includes a LC variable region (LCVR) and a LC constant region (LCCR). Here, the Ab is an immunoglobulin G (IgG) type Ab, and the IgG isotype may be further divided into subclasses (e.g., IgG1, IgG2, IgG3 and IgG4). The HCVR and LCVR regions can be further subdivided into regions of hyper-variability, termed complementarity determining regions (CDRs), interspersed with regions that are more conserved, termed framework regions (FR). Each HCVR and LCVR includes three CDRs and four FRs, arranged from N-terminus to C-terminus in the following order: FR1, CDR1, FR2, CDR2, FR3, CDR3, FR4. Herein, the three CDRs of the HC are referred to as HCDR1, HCDR2 and HCDR3, and the three CDRs of the LC are referred to as LCDR1, LCDR2 and LCDR3. The CDRs contain most of the residues that form specific interactions with an antigen, such as 2019-nCOV RBD. Assigning the residues to the various CDRs may be done by algorithms such as, for example, Chothia, Kabat or North. The North CDR definition is based on affinity propagation clustering with a large number of crystal structures (North et al. (2011) J. Mol. Bio. 406:228-256). Herein, the CDRs are best defined by the sequences listed in the Sequence Listing, which are based upon a combination of multiple definitions including North.

As used herein, “acute respiratory syndrome coronavirus 2,” “SARS-COV-2,” “2019 novel coronavirus,” “2019-nCOV” and the like mean the respiratory virus that causes COVID-19, which typically is characterized by mild to severe lower respiratory tract disease.

Like all coronavirus, 2019-nCOV has four structural proteins, including spike (S), envelope (E), membrane (M), and nucleocapsid (N) proteins. Among them, S protein plays an important role in viral attachment, fusion and entry, and it serves as a target for developing Abs, entry inhibitors and vaccines. S protein mediates viral entry into host cells by first binding to a host receptor through the RBD in the S1 subunit and then fusing the viral and host membranes through the S2 subunit.

Based on the research of other coronaviruses, especially SARS-COV and MERS-CoV, the important envelope protein that binds to the receptor is the S protein. The S protein can be further divided to S1 and S2. The function of S2 is to mediate membrane fusion. As described herein, the C-terminal domain (CTD) is identified as 2019-nCOV RBD, which binds to the ACE2 receptor. Therefore, Abs that target RBD and Abs that block the binding of S to ACE2, may become the neutralizing Abs that attenuate or inhibit 2019-nCOV infection.

As used herein, “bind” or “binds” means an ability of a protein to form a type of chemical bond or attractive force with another protein or molecule as determined by common methods known in the art. Binding can be characterized by an equilibrium dissociation constant (K_{D}) of about 1×10⁻⁶ M or less (i.e., a smaller K_{D} denotes a tighter binding). Methods of determining whether two molecules bind are well known in the art and include, for example, equilibrium dialysis, surface plasmon resonance, and the like. Ab binding can be determined in standard ELISA assays in a single point format, as described below and binding may be characterized by, for example, a Biacore® analyzer (GE Healthcare Bio-Sciences AB; Uppsala, Sweden), as described below. While the Abs herein are human, they may, however, exhibit cross-reactivity to other species.

As used herein, “biological activity,” with regard to a monoclonal Ab, means an ability of the Ab to bind to an antigen. It also can include Ab binding to antigen and resulting in a measurable biological response that can be measured in vitro or in vivo. Such activity may be antagonistic or agonistic.

As used herein, “buffer” means a buffered solution that resists changes in pH by the action of its acid-base conjugate components. One buffer is a histidine buffer, which is a buffer including histidine ions. Examples of histidine buffers include, but are not limited to, histidine acetate, histidine chloride, histidine phosphate, histidine succinate, histidine sulfate, etc.

As used herein, “chemical stability” means an ability of a therapeutic agent, substance or product to resist potential changes in composition in the product due to chemical reactions that may occur, such as isomerization, aggregation, oxidation, polymerization, fragmentation, and hydrolysis.

As used herein, “2019-nCOV antibody,” 2019-nCOV Ab” and the like, means an Ab that binds to the RBD of 2019-nCOV, thereby blocking its binding to an angiotensin-converting enzyme 2 (ACE2) receptor. An exemplary 2019-nCov Ab is etesevimab, which is an IgG1 Ab that binds to 2019-nCOV RBD and blocks binding between 2019-nCOV RBD and the ACE2 receptor, thereby inhibiting 2019-nCOV infection. An exemplary 2019-nCOV Ab includes three HCDRs—HCDR1, HCDR2 and HCDR3—and includes three LDCRs—LCDR1, LCDR2 and LCDR3—where HCDR1 has the amino acid sequence of SEQ ID NO:1, HCDR2 has the amino acid sequence of SEQ ID NO:2, HCDR3 has the amino acid sequence of SEQ ID NO:3, LCDR1 has the amino acid sequence of SEQ ID NO:4, LCDR2 has the amino acid sequence of SEQ ID NO:5 and LCDR3 has the amino acid sequence of SEQ ID NO:6. Alternatively, the 2019-nCOV Ab includes a HCVR having the amino acid sequence of SEQ ID NO:7. Alternatively still, the 2019-nCOVAb includes a LCVR having the amino acid sequence of SEQ ID NO:8. Alternatively still, the 2019-nCOV Ab includes a HCVR having the amino acid sequence of SEQ ID NO:7 and a LCVR having the amino acid sequence of SEQ ID NO:8. Alternatively, the 2019-nCOV Ab includes a HC having the amino acid sequence of SEQ ID NO:9. Alternatively still, the 2019-nCOV Ab includes a LC having the amino acid sequence of SEQ ID NO:10. Alternatively still, the 2019-nCOV Ab includes a HC having the amino acid sequence of SEQ ID NO:9 and a LC having the amino acid sequence of SEQ ID NO: 10. Specifically, etesevimab is a human IgG1 variant (Fc effector null) monoclonal Ab having 2 identical HC polypeptides of 449 amino acids (SEQ ID NO:9) and 2 identical LC polypeptides of 216 amino acids (SEQ ID NO:10) each produced using a Chinese hamster ovary (CHO) cell line. Post-translational modifications of etesevimab include N-linked glycosylation on HC Asn297 and removal of a HC C-terminal Lys.

As used herein, “effective amount” means an amount, concentration or dose of one or more active ingredients (e.g., an Ab), or a pharmaceutically acceptable salt thereof that, upon single or multiple dose administration to an individual in need thereof, provides a desired effect in such an individual under diagnosis or treatment (i.e., may produce a clinically measurable difference in a condition of the individual). An effective amount can be readily determined by one of skill in the art by using known techniques and by observing results obtained under analogous circumstances. In determining the effective amount for an individual, a number of factors are considered, including, but not limited to, the species of mammal, its size, age and general health, the specific disease or disorder involved, the degree of or involvement or the severity of the disease or disorder, the response of the individual, the particular incretin analog administered, the mode of administration, the bioavailability characteristics of the preparation administered, the dose regimen selected, the use of concomitant medication, and other relevant circumstances.

As used herein, “frozen,” with regard to a formulation, means one at a temperature below 0° C. Generally, a frozen formulation is not freeze-dried, nor is it subjected to prior, or subsequent, lyophilization. In this manner, a frozen formulation includes frozen active ingredient (e.g., an Ab) for storage (e.g., in stainless steel tank) or frozen drug product (in final vial configuration).

As used herein, “individual in need thereof” means a mammal, such as a human, with a condition, disease, disorder or symptom requiring treatment or therapy, including for example, those listed herein. In particular, the preferred individual to be treated is a human, especially an individual having or suspected of having a 2019-nCOV infection or COVID-19.

As used herein, “kit” means any manufacture (e.g., a package or container) including at least one reagent (e.g., a medicament) for treatment of COVID-19 and a package insert having instructions for administering the same to an individual having a disease, disorder or condition associated with COVID-19. The manufacture can be promoted, distributed or sold as a unit for performing the methods herein.

As used herein, “medicament” means an active ingredient (e.g., an Ab) to treat a disease, disorder and/or condition such as COVID-19.

As used herein, “microbiological stability” means an ability of an active ingredient, substance or product to maintain its sterility when exposed to environmental or other microorganisms.

As used herein, “pharmaceutical formulation” or “formulation” means a preparation that is in such form as to permit the biological activity of the active ingredient (e.g., an Ab) to be effective and that contains no additional components having unacceptable toxicity to an individual to which the formulation would be administered. Such formulations are sterile. “Pharmaceutically acceptable” excipients (e.g., additives, vehicles, etc.) mean those that reasonably can be administered to an individual to provide an effective dose of the active ingredient employed.

As used herein, “treat,” “treatment” or “treating” means managing and caring for an individual having or suspected of having a condition for which formulation administration is indicated for the purpose of combating or alleviating symptoms and complications of its infection. Treating includes administering a compound, composition or formulation including the active ingredient herein (e.g., an Ab) to such an individual to prevent the onset of symptoms or complications, alleviating the symptoms or complications, or eliminating the infection. Treating includes administering a compound, composition or formulation including the active ingredient herein (e.g., an Ab) to an individual in need thereof to result in an attenuated or blocked 2019-nCOV infection. The individual to be treated is an animal, especially a human being.

As used herein, “patient,” “subject” and “individual,” are used interchangeably herein, and mean an animal, especially a human. In certain instances, the individual is a human and is further characterized with an infection that would benefit from administration of the formulation herein.

As used herein, “polyol” means a substance with multiple hydroxyl groups that can be included in a formulation and includes sugars (reducing and non-reducing sugars), sugar alcohols and sugar acids. Examples of reducing sugars include, but are not limited to, fructose, mannose, maltose, lactose, arabinose, xylose, ribose, rhamnose, galactose and glucose. Examples of non-reducing sugars include, but are not limited to, sucrose, trehalose, sorbose, melezitose and raffinose. Examples of sugar alcohol include, but are not limited to, mannitol, xylitol, erythritol, threitol, sorbitol and glycerol. Examples of sugar acids include, but are not limited to, L-gluconate and metallic salts thereof. In some instances, the polyol is a non-reducing sugar or a sugar alcohol, especially sucrose or mannitol.

As used herein, “preservative” means a compound that can be included in a formulation to essentially reduce bacterial action therein, thus facilitating the production of a multi-use formulation. Examples of preservatives include, but are not limited to, octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other preservatives include aromatic alcohols such as phenol, butyl and benzyl alcohol, alkyl parabens such as methyl or propyl paraben, catechol, resorcinol, cyclohexanol, 3-pentanol, and m-cresol.

As used herein, “stable,” with regard to a formulation, means one in which the active ingredient therein (e.g., an Ab) essentially retains its biological activity and/or chemical stability and/or physical stability upon storage. In this manner, the formulation essentially retains its chemical and physical stability, as well as retain its biological activity upon storage. The storage period generally can be based on an intended shelf-life of the formulation.

As used herein, “sterile,” with regard to a formulation, means aseptic or free or essentially free from all living microorganisms and spores.

As used herein, “surfactant” means a surface-active agent, typically a nonionic surfactant. Examples of surfactants include, but are not limited to, polysorbate (e.g., polysorbate 20, 40, 60 and 80); poloxamer (e.g., poloxamer 188); Triton; sodium dodecyl sulfate (SDS); sodium laurel sulfate; sodium octyl glycoside; lauryl-, myristyl-, linoleyl- or stearyl-sulfobetaine; lauryl-, myristyl-, linoleyl- or stearyl-sarcosine; linoleyl-, myristyl- or cetyl-betaine; lauroamidopropyl-, cocamidopropyl-, linoleamidopropyl-, myristamidopropyl-, palmidopropyl- or isostearamidopropyl-betaine (e.g., lauroamidopropyl); myristamidopropyl-, palmidopropyl- or isostearamidopropyl-dimethylamine; sodium methyl cocoyl- or disodium methyl oleyl-taurate; and the MONAQUAT™ series (Mona Industries, Inc.; Paterson, N.J.); polyethyl glycol, polypropyl glycol, and copolymers of ethylene and propylene glycol (e.g., Pluronics, PF68, etc.); etc., especially polysorbate 80.

### Compositions and Formulations

The compositions herein are formulations that include a 2019-nCOV Ab having three HCDRs (i.e., HCDR1, HCDR2 and HCDR3) and having three LDCRs (i.e., LCDR1, LCDR2 and LCDR3), where HCDR1 has the amino acid sequence of SEQ ID NO:1, HCDR2 has the amino acid sequence of SEQ ID NO:2, HCDR3 has the amino acid sequence of SEQ ID NO:3, LCDR1 has the amino acid sequence of SEQ ID NO:4, LCDR2 has the amino acid sequence of SEQ ID NO:5 and LCDR3 has the amino acid sequence of SEQ ID NO:6. Alternatively, the formulations can include a 2019-nCOV Ab having a HCVR having the amino acid sequence of SEQ ID NO:7 and having a LCVR having the amino acid sequence of SEQ ID NO:8. Alternatively, the formulations can include a 2019-nCOV Ab having a HC having the amino acid sequence of SEQ ID NO:9 and having a LC having the amino acid sequence of SEQ ID NO: 10. Further details on 2019-nCOV Abs, including methods of synthesizing the same, are described in Intl. Patent Application Publication No. WO 2021/169932.

In some instances, the 2019-nCOV Ab can include a HCVR sequence having at least about 90%, 91%, 92%, 93%, 94%, 95%, 96%, 97%, 98%, 99% or 100% sequence identity to the amino acid sequence of SEQ ID NO: 7. In other instances, the 2019-nCOV Ab can include a LCVR sequence having at least about 90%, 91%, 92%, 93%, 94%, 95%, 96%, 97%, 98% or 99% identity to the amino acid sequence of SEQ ID NO:8. The HCVR and/or LCVR can include substitutions (e.g., conservative substitutions), insertions or deletions relative to the reference sequence but retains an ability to bind to 2019-nCOV RBD. That is, a total of 1 to 10 amino acids can be substituted, inserted and/or deleted in SEQ ID NOS:7 and 8. In some instances, substitutions, insertions or deletions occur in regions outside the CDRs (i.e., in the framework regions (FRs)).

The 2019-CoV Ab can be at a concentration from about 25 mg/mL to about 150 mg/mL. In some instances, the 2019-nCOV Ab is at a concentration of about 30 mg/mL to 145 mg/mL, about 35 mg/mL to about 140 mg/mL, about 40 mg/mL to about 135 mg/mL, about 45 mg/mL to about 130 mg/mL, about 50 mg/mL to about 125 mg/mL, about 55 mg/mL to about 120 mg/mL, about 60 mg/mL to about 115 mg/mL, about 65 mg/mL to about 110 mg/mL, about 70 mg/mL to about 105 mg/mL, about 75 mg/mL to about 100 mg/mL, about 80 mg/mL to about 95 mg/mL, or about 85 mg/mL to about 90 mg/mL. In some instances, the 2019-nCOV Ab can be at a concentration of about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, or about 150 mg/mL, especially from about 30 mg/mL to about 40 mg/mL. In certain instances, the 2019-nCOV Ab is at a concentration of 35 mg/mL.

In addition to the 2019-nCOV Ab, the formulations also include a buffer system to maintain a proper pH. An exemplary buffer system is a histidine buffer, which can be L-histidine and/or L-histidine hydrochloride monohydrate.

The histidine buffer can be at a concentration from about 5 mM to about 40 mM. In some instances, the histidine buffer can be at a concentration from about 10 mM to about 35 mM, about 15 mM to about 30 mM, or about 20 mM to about 25 mM. In some instances, the histidine buffer can be at a concentration of about 5 mM, about 10 mM, about 15 mM, about 20 mM, about 25 mM, about 30 mM, about 35 mM, or about 40 mM, especially from about 15 mM to about 25 mM. In certain instances, the histidine buffer is at a concentration of 20 mM.

Regardless of the buffer system, the pH of the formulations herein can be from about 5.5 to about 7.0. In some instances, the pH can be from about 5.6 to about 6.9, from about 5.7 to about 6.8, from about 5.8 to about 6.7, from about 5.9 to about 6.6, from about 6.0 to about 6.5, from about 6.1 to about 6.4, or from about 6.2 to about 6.3. In some instances, the pH can be about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, or about 7.0. In certain instances, the pH is 6.0.

In addition to the above, the formulations herein also include a polyol or tonicity agent to render an isotonic formulation, which is suitable for intravenous or subcutaneous administration. Exemplary polyol or tonicity agents are mannitol or sucrose.

When the polyol or tonicity agent is mannitol, it can be at a concentration from about 3.5% (w/v) to about 5.5% (w/v). In some instances, mannitol can be at a concentration from about 3.6% (w/v) to about 5.4% (w/v), about 3.7% (w/v) to about 5.3% (w/v), about 3.8% (w/v) to about 5.2% (w/v), about 3.9% (w/v) to about 5.1% (w/v), about 4.0% (w/v) to about 5.0% (w/v), about 4.1% (w/v) to about 4.9% (w/v), about 4.2% (w/v) to about 4.8% (w/v), about 4.3% (w/v) to about 4.7% (w/v), about 4.4% to about 4.6% (w/v), or about 4.5% (w/v). In some instances, mannitol can be a concentration of about 3.5% (w/v), about 3.6% (w/v), about 3.7% (w/v), about 3.8% (w/v), about 3.9% (w/v), about 4.0% (w/v), about 4.1% (w/v), about 4.2% (w/v), about 4.3% (w/v), about 4.4% (w/v), about 4.5% (w/v), about 4.6% (w/v), about 4.7% (w/v), about 4.8% (w/v), about 4.9% (w/v), about 5.0% (w/v), about 5.1% (w/v), about 5.2% (w/v), about 5.3% (w/v), about 5.4% (w/v), or about 5.5% (w/v). In certain instances, mannitol is at a concentration of 4.5% (w/v).

When the polyol or tonicity agent is sucrose, it can be at a concentration from about 6.5% (w/v) to about 8.5% (w/v). In some instances, sucrose can be at a concentration from about 6.6% (w/v) to about 8.4% (w/v), about 6.7% (w/v) to about 8.3% (w/v), about 6.8% (w/v) to about 8.2% (w/v), about 6.9% (w/v) to about 8.1%, about 7.0% (w/v) to about 8.0% (w/v), about 7.1% (w/v) to about 7.9% (w/v), about 7.2% (w/v) to about 7.8% (w/v), about 7.3% (w/v) to about 7.7% (w/v), about 7.4% (w/v) to about 7.6% (w/v), or about 7.5% (w/v). In some instances, sucrose can be at a concentration of about 6.5% (w/v), about 6.6% (w/v), about 6.7% (w/v), about 6.8% (w/v), about 6.9% (w/v), about 7.0% (w/v), about 7.1% (w/v), about 7.2% (w/v), about 7.3% (w/v), about 7.4% (w/v), about 7.5% (w/v), about 7.6% (w/v), about 7.7% (w/v), about 7.8% (w/v), about 7.9% (w/v), about 8.0% (w/v), about 8.1% (w/v), about 8.2% (w/v), about 8.3% (w/v), about 8.4% (w/v), or about 8.5% (w/v). In some instances, sucrose can be at a concentration of about 8.00% (w/v), about 8.01% (w/v), about 8.02% (w/v), about 8.03% (w/v), about 8.04% (w/v), about 8.05% (w/v), about 8.06% (w/v), about 8.07% (w/v), about 8.08% (w/v), about 8.09% (w/v), or about 8.10% (w/v). In certain instances, sucrose is at a concentration of 8.04% (w/v).

In addition to the above, the formulations herein also include a surfactant to improve physical and chemical characteristics of the formulations and to improve efficacy or bioperformance thereof.

In some instances, the surfactant can be a non-ionic surfactant. Exemplary non-ionic surfactants include, but are not limited to, polysorbates (e.g., polysorbate 20, 40, 60, 80, etc.) or poloxamers (e.g., poloxamer 184, 188, etc.), especially polysorbate 80.

When the surfactant is polysorbate 80, it can be at a concentration from about 0.01% (w/v) to about 0.1% (w/v). In some instances, polysorbate 80 can be at a concentration from about 0.02% (w/v) to about 0.09% (w/v), 0.03% (w/v) to about 0.08% (w/v), about 0.04% (w/v) to about 0.07% (w/v), or about 0.05% (w/v) to about 0.06% (w/v). In some instances, polysorbate 80 can be at a concentration of about 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), or 0.1% (w/v). In certain instances, polysorbate 80 is at a concentration of 0.05% (w/v).

The formulations herein are sterile when first produced. As such, the formulations optionally can include a preservative that is compatible with the other components of the composition and that may be added at sufficient strength to meet applicable regulatory anti-microbial preservative requirements. Pharmaceutically acceptable preservatives are known to one of skill in the art (see, e.g., Remington: The Science and Practice of Pharmacy (Troy, Ed., 21^{st} Edition, Lippincott, Williams & Wilkins, 2006). In some instances, the formulations herein are preservative-free.

In one particular instance, the formulation includes 2019-nCOV Ab at a concentration of about 35 mg/mL in a L-histidine buffer at a concentration of about 20 mM at pH 6.0, with sucrose at a concentration of about 8.04% (w/v) and polysorbate 80 at a concentration of about 0.05% (w/v), where the 2019-nCOV Ab includes a HCDR1 having the amino acid sequence of SEQ ID NO: 1, a HCDR2 having the amino acid sequence of SEQ ID NO:2, a HCDR3 having the amino acid sequence of SEQ ID NO:3, a LCDR1 having the amino acid sequence of SEQ ID NO:4, a LCDR2 having the amino acid sequence of SEQ ID NO:5, and a LCDR3 having the amino acid sequence of SEQ ID NO:6. In another particular instance, the formulation includes 2019-nCOV Ab at a concentration of about 35 mg/mL in a L-histidine buffer at a concentration of about 20 mM at pH 6.0, with sucrose at a concentration of about 8.04% (w/v) and polysorbate 80 at a concentration of about 0.05% (w/v), where the 2019-nCOV Ab includes a HCVR having the amino acid sequence of SEQ ID NO:7 and a LCVR having the amino acid sequence of SEQ ID NO:8. In yet another particular instance, the formulation includes 2019-nCOV Ab at a concentration of about 35 mg/mL in a L-histidine buffer at a concentration of about 20 mM at pH 6.0, with sucrose at a concentration of about 8.04% (w/v) and polysorbate 80 at a concentration of about 0.05% (w/v), where the 2019-nCOV Ab includes a HC having the amino acid sequence of SEQ ID NO:9 and a LC having the amino acid sequence of SEQ ID NO:10.

The compositions herein can be administered intravenously (IV), intramuscularly (IM) or subcutaneously (SQ). The compositions can be administered using a pre-filled, disposable pen, reusable pen, or automatic pen injector. Alternatively, the compositions may be administered using a multi-use vial or a pump device. In some instances, the device is an automatic injection apparatus as described in U.S. Pat. No. 8,734,394.

The compositions herein therefore may be presented in a pre-filled syringe/multi-use vial. Such pre-filled syringe/multi-use vial may be useful for administering about 0.5 mL to about 1 mL of the composition per patient per dose. The dose of the composition may be administered using a dosing schedule determined by a clinician, physician or other trained medical professional.

Alternatively, the composition can be prepared for a cartridge and therefore will differ from the above composition by including a preservative.

Alternatively, the composition can be prepared as part of an article of manufacture comprising the composition, where the article of manufacture can be a multi-use vial, a reusable pen injector, a pre-filled, disposable pen, an autoinjector or a pump.

In view of the above, the compositions herein are associated with acceptable shelf-life stability, in-use stability and acceptable injection site experience.

### Methods

The formulations herein may be used for attenuating, preventing and/or treating 2019-nCoV infection or COVID-19. For example, the formulations herein may be used for treating an individual having an active 2019-nCoV infection. In particular, methods are provided for treating such an infected individual, where such methods include at least a step of administering to an individual in need of such treatment an effective amount of a formulation herein. In addition, methods are provided for prophylactically preventing infection in an individual, where such methods include at least a step of administering to an individual at risk for exposure to 2019-nCOV an effective amount of a formulation herein.

In these methods, effectiveness of the composition can be assessed by, for example, observing a significant reduction in ageusia, anorexia, cough, diarrhea, dysgeusia, fever, headaches, joint pain, muscle pain, nausea, shortness of breath, sore throat and/or vomiting.

### EXAMPLES

The following non-limiting examples are offered for purposes of illustration, not limitation.

### Formulations

### Example 1: Formulations Including a 2019-nCOV Ab

The formulations are prepared substantially as described herein. Such compositions include a 2019-nCOV Ab such as etesevimab (e.g., HC of SEQ ID NO:9 and LC of SEQ ID NO:10) at 35, 40 or 125 mg/mL and additional ingredients as set forth in Table 1. Polyol concentrations are selected to render an isotonic formulation suitable for IV or SQ administration.

Solutions are prepared by adding the 2019-nCOV Ab into an appropriate matrix, mixing until dissolution into a solution is achieved, and then the solution is brought to final volume using appropriately sized mixing vessel. Each formulation solution is aseptically filtered into 0.22-μm PVDF filters and is filled into type 1 glass vials, at a predetermined fill volume.

TABLE 1
Exemplary Formulations.

| For- | | | | | 2019- |
|---|---|---|---|---|---|
| mula- | | | | | nCoV |
| tion | | Buffer | Surfactant | Polyol | Ab |
| No. | pH | (mM) | (% (w/v)) | (% (w/v)) | (mg/mL) |
| 1 | 6 | L-His; 20 | polysorbate 80; 0.02 | mannitol; 4.5 | 40 |
| 2 | 6 | L-His; 20 | polysorbate 80; 0.05 | sucrose; 8.04 | 35 |
| 3 | 6 | L-His; 20 | polysorbate 80; 0.05 | sucrose; 8.04 | 125 |
| 4 | 6 | L-His; 20 | polysorbate 80; 0.02 | sucrose; 8.04 | 40 |
| 5 | 6 | L-His; 20 | polysorbate 80; 0.05 | sucrose; 8.04 | 150 |
| 6 | 6 | L-His; 20 | polysorbate 80; 0.02 | trehalose: 8.2 | 40 |
| 7 | 6 | L-His; 20 | polysorbate 80; 0.05 | sucrose; 8.5 | 35 |

For Formulation 1, the 2019-nCOV Ab is provided as a 400 mg/10 mL vial and is supplied as a sterile refrigerated solution intended for IV administration. Alternatively, the 2019-nCOV Ab is provided as a 700 mg/17.5 mL vial and is supplied as a sterile refrigerated solution intended for IV administration.

For Formulation 2, the 2019-nCOV Ab is provided as a 700 mg/20 mL vial and is supplied as a sterile refrigerated solution intended for IV administration.

For Formulation 3, the 2019-nCOV Ab is provided as a 262.5 mg/2.1 mL vial is supplied as a sterile frozen solution intended for IV administration and/or SC administration.

For Formulation 4, the 2019-nCOV Ab is evaluated for stability but not provided for clinical trials.

For Formulation 5, the 2019-nCOV Ab is evaluated for stability and used as drug substance formulation.

For Formulation 6, the 2019-nCOV Ab is evaluated for stability but not provided for clinical trials.

For Formulation 7, the 2019-nCOV Ab is evaluated for stability but not provided for clinical trials.

Formulations 1 and 2 are stored at 2° C.-8° C., while Formulation 3 is stored at not more than −40° C. or alternatively at 2° C.-8° C., for further studies as described in the subsequent Examples.

### In Vitro Data (Chemical and Physical Stability)

### Example 2: In-Use Stability Studies

Stability-indicating analytical and characterization techniques selected to measure the chemical and physical stability of the formulations include size exclusion chromatography (SEC) and visual appearance.

TABLE 2
Freeze/Thaw Stability of Formulations 1, 4 and 6.

| | | Formulation | | | | | |
|---|---|---|---|---|---|---|---|
| | | (40 mg/mL 2019-nCoV Ab in 20 mM L-histidine buffer, | | | | | |
| | | pH 6.0, 0.02% (w/v) polysorbate 80) | | | | | |
| | | w/ 4.5% (w/v) | | w/ 8.04% (w/v) | | w/ 8.2% (w/v) | |
| | | mannitol | | sucrose | | trehalose | |
| | | (Formulation 1) | | (Formulation 4) | | (Formulation 6) | |
| | | SEC | SEC | SEC | SEC | SEC | SEC |
| Freeze/Thaw | | Total | Total | Total | Total | Total | Total |
| Temp. | No. of | Aggregates | Monomer | Aggregates | Monomer | Aggregates | Monomer |
| (° C.) | Cycles | (%) | (%) | (%) | (%) | (%) | (%) |
| −40° C./RT¹ | 0 | 0.7 | 99.3 | 0.7 | 99.3 | 0.7 | 99.3 |
| | 3 | 3.1 | 96.8 | 0.7 | 99.2 | 0.7 | 99.2 |
| −80° C./RT² | 0 | 0.7 | 99.3 | 0.7 | 99.3 | 0.7 | 99.3 |
| | 3 | 4.5 | 95.4 | 0.9 | 99.0 | 0.7 | 99.2 |

NOTE:
¹prepared as 30 mL fill in 50 mL ultra-low density polyethylene (ULDP) bag;
²prepared as 60 mL fill in 125 mL polycarbonate (PC) bottle.

As sucrose demonstrates capability to protect 2019-nCOV Ab against freeze/thaw stress, another formulation (i.e., Formulation 5) is generated to evaluate frozen stability at a higher concentration of 150 mg/mL in formulation of 20 mM L-histidine buffer, pH 6, 8.04% (w/v) sucrose, and 0.05% (w/v) polysorbate 80 (Table 3).

TABLE 3
Freeze/Thaw Stability of Formulation 5.
Formulation
(150 mg/mL 2019-nCoV Ab in 20 mM L-histidine buffer,
pH 6.0, 8.04% (w/v) sucrose, 0.05% (w/v) polysorbate 80)

| Study | Temperature | | Initial (T₀) | 1-Month |
|---|---|---|---|---|
| UV Content (mg/mL) | −70° | C. | 149.4 | 145.2 |
| | −40° | C. | | 149.4 |
| | 5° | C. | | 149.7 |
| SEC Monomer | −70° | C. | 98.7 | 98.7 |
| Purity (%) | | | 1.3 | 1.3 |
| Total Aggregates (%) | −40° | C. | | 98.5 |
| | | | | 1.5 |
| | 5° | C. | | 98.7 |
| | | | | 1.3 |
| Visual Appearance | −70° | C. | | no change |
| | −40° | C. | | no change |
| | 5° | C. | slightly opalescent, | no change |
| | | | slightly yellow liquid | |
| | | | w/ no particles | |

NOTE:
prepared as 2.5 mL fill in 5 mL PC bottle

The stability results show that the 2019-nCOV Ab is suitable to be formulated at 150 mg/mL in 20 mM L-histidine buffer, pH 6, 8.04% (w/v) sucrose, and 0.05% (w/v) polysorbate 80.

Impact of long-term storage at 2° C.-8° C. and accelerated conditions at 25° C. are tested on a formulation having 35 mg/mL of 2019-nCOV Ab in 20 mM L-histidine buffer, pH 6, 8.5% (w/v) sucrose, and 0.05% (w/v) polysorbate 80 (i.e., Formulation 7; Table 4) compared to a formulation having 40 mg/mL of 2019-nCOV Ab in 20 mM L-histidine buffer, pH 6.0, 4.5% (w/v) mannitol, and 0.02% (w/v) polysorbate 80 (Formulation 1; Table 5).

TABLE 4
Stability of Formulations Having 2019-nCoV Ab.

| | | | Formulation | |
|---|---|---|---|---|
| | | | (35 mg/mL 2019-nCoV Ab in 20 mM | |
| | | | L-histidine buffer, pH 6.0, 8.5% (w/v) | |
| | | | sucrose, 0.05% (w/v) polysorbate 80) | |
| Analytical | | Storage | Months | |
| Procedure | Unit | Condition | 0 | 1 |
| Quantity (UV) | mg/mL | 2° C.-8° C. | 35.9 | 34.5 |
| | | 25° C. | | 33.8 |
| Monomer Purity | % | 2° C.-8° C. | 98.1 | 98.2 |
| (SEC) | | 25° C. | | 98.1 |
| Total | % | 2° C.-8° C. | 1.8 | 1.8 |
| Aggregates | | 25° C. | | 1.7 |
| (SEC) | | | | |
| Purity (reduced) | % | 2° C.-8° C. | 97.7 | 97.5 |
| (reduced CE- | | 25° C. | | 97.3 |
| SDS) | | | | |
| Purity (non- | % | 2° C.-8° C. | 97.8 | 97.1 |
| reduced) | | 25° C. | | 97.0 |
| (non-Reduced | | | | |
| CE-SDS) | | | | |
| Description | N/A | 2° C.-8° C. | Colorless, clear | Colorless, w/ a slight |
| (visual) | | | and no visible | opalescence and no |
| | | | particles | visible particles |
| | | 25° C. | | Colorless, w/ a slight |
| | | | | opalescence and no |
| | | | | visible particles |
| Charge | % | 2° C.-8° C. | 83.0 | 82.8 |
| Heterogeneity | | | 13.9 | 13.2 |
| (CEX) | | | 3.1 | 4.0 |
| Main Peak | | 25° C. | | 80.1 |
| Total Acidic | | | | 15.3 |
| Variants | | | | 4.7 |
| Total Basic | | | | |
| Variants | | | | |
| Particular | particles/mL | 2° C.-8° C. | 3 | 65 |
| Matter (HIAC) | | | 0 | 19 |
| ≥10 μm | | 25° C. | | 19 |
| ≥25 μm | | | | 3 |

TABLE 5
Stability of Formulations Having 2019-nCoV Ab.

| | | | Formulation | |
|---|---|---|---|---|
| | | | (40 mg/mL 2019-nCoV Ab in 20 mM | |
| | | | L-histidine buffer pH 6.0, 4.5% (w/v) | |
| | | | mannitol, and 0.02% (w/v) polysorbate 80) | |
| Analytical | | Storage | Months | |
| Procedure | Unit | Condition | 0 | 1 |
| Quantity (UV) | mg/mL | 2° C.-8° C. | 39.7 | 39.6 |
| | | 25° C. | | 39.6 |
| Monomer Purity | % | 2° C.-8° C. | 99.3 | 99.3 |
| (SEC) | | 25° C. | | 99.2 |
| Total | % | 2° C.-8° C. | 0.6 | 0.6 |
| Aggregates | | 25° C. | | 0.7 |
| (SEC) | | | | |
| Purity (reduced) | % | 2° C.-8° C. | 98.3 | 98.4 |
| (reduced CE- | | 25° C. | | 98.4 |
| SDS) | | | | |
| Purity (non- | % | 2° C.-8° C. | 97.9 | 97.8 |
| reduced) | | 25° C. | | 97.6 |
| (non-Reduced | | | | |
| CE-SDS) | | | | |
| Description | N/A | 2° C.-8° C. | Colorless, w/ a | Colorless, w/ a slight |
| (visual) | | | slight | opalescence and no |
| | | | opalescence and | visible particles |
| | | 25° C. | no visible | Colorless, w/ a slight |
| | | | particles | opalescence and no |
| | | | | visible particles |
| Charge | % | 2° C.-8° C. | 84.0 | 83.6 |
| Heterogeneity | | | 12.9 | 13.6 |
| (CEX) | | | 3.0 | 2.8 |
| Main Peak | | 25° C. | | 81.1 |
| Total Acidic | | | | 15.5 |
| Variants | | | | 3.4 |
| Total Basic | | | | |
| Variants | | | | |
| Particular | particles/mL | 2° C.-8° C. | 9 | 2 |
| Matter (HIAC) | | | 3 | 0 |
| ≥10 μm | | 25° C. | | 1 |
| ≥25 μm | | | | 0 |

Stability of the formulation containing sucrose (Table 4) demonstrates a comparable stability to the formulation containing mannitol (Table 5), indicating that replacing mannitol with sucrose does not impact stability of 2019-nCOV Ab under long-term storage (2° C.-8° C.) and accelerated (25° C.) conditions. To ensure the formulation is isotonic for IV injection, the amount of sucrose is reduced from 8.5% (w/v) to 8.04% (w/v). Therefore, one formulation contains 35 mg/mL of 2019-nCOV in 20 mM L-histidine buffer, pH 6, 8.04% (w/v) sucrose, 0.05% (w/v) polysorbate 80, and water for injection (i.e., Formulation 2).

Additional studies are performed on Formulations 1 and 2 (Table 6).

TABLE 6
Formulation 1 and 2 Comparability Studies.

| | Analytical | Formulation 1 | | Formulation 2 |
|---|---|---|---|---|
| Parameter | Procedure | Batch 1 | Batch 2 | Batch 1 |

Biological Activity

| | | | | |
|---|---|---|---|---|
| Potency (ACE2 | ELISA | 96 | 94 | 111 |
| blocking; %) | | | | |

Molecular Heterogeneity

| | | | |
|---|---|---|---|
| Main Peak (%) | 79.8 | 81.4 | 75.0 |
| Total Acidic | 14.0 | 14.7 | 17.2 |
| Variants (%) | | | |
| Total Basic | 6.2 | 3.9 | 7.7 |
| Variants (%) | | | |

Purity: Product-Related Impurities

| | | | | |
|---|---|---|---|---|
| Monomer Purity | Size-exclusion | 99.3 | 99.4 | 99.2 |
| (%) | chromatography | | | |
| Total Aggregates | (SEC) | 0.7 | 0.5 | 0.8 |
| (%) | | | | |
| Purity (reduced; %) | CE-SDS | 97.1 | 97.3 | 97.8 |
| Total Fragments | (reduced) | 1.8 | 1.4 | 1.5 |
| (reduced; %) | | | | |
| Purity | CE-SDS | 97.5 | 97.0 | 98.4 |
| (non-reduced; %) | (non-reduced) | | | |
| Total Fragments | | 2.5 | 3.0 | 1.6 |
| (non-reduced; %) | | | | |

Other

| | | | | |
|---|---|---|---|---|
| Description | Visual | Slightly | Colorless, | Slightly yellow, slightly |
| | | yellow, | slightly | opalescent solution, free |
| | | slightly | opalescent | of visible particles |
| | | opalescent | solution, | |
| | | solution, | free of | |
| | | free of | visible | |
| | | visible | particles | |
| | | particles | | |
| pH | USP <791>, | 6.2 | 6.2 | 6.1 |
| | Ph. Eur. 2.2.3, | | | |
| | JP 2.54 | | | |
| Polysorbate 80 (%) | HPLC-UV | 0.0200 | 0.0200 | 0.049 |
| Particulate Matter | USP <787> | | | |
| (particles/container) | | | | |
| ≥10 μm | | 163 | 120 | 73 |
| ≥25 μm | | 0 | 0 | 0 |
| Subvisible Particles | HIAC | | | |
| (particles/container) | | | | |
| ≥2 μm | | 3471 | 1747 | 2447 |
| ≥5 μm | | 805 | 407 | 540 |
| Subvisible Particles | MFI | | | |
| (particles/mL) | | | | |
| ≥2 μm | | 673 | 292 | 639 |
| ≥5 μm | | 108 | 48 | 138 |
| Osmolality | USP <785> | 292 | 295 | 293 |
| (mOsm/kg) | | | | |

In-use compatibility also is assessed on Formulation 2, which is administered neat (undiluted) or is further diluted with 0.9% Sodium Chloride Injection, as appropriate, to evaluate compatibility with, for example, contact materials of dosing devices representing that used in clinical sites and the molecule stability during dose preparation and administration. The contact materials tested are polyvinylchloride (PVC), polyolefin (polypropylene and polyethylene) and polyethersulfone (PES).

Likewise, simulated infusion studies are conducted to evaluate compatibility with an IV bag and infusion set made from PVC and in-line filter made from PES. The formulation is prepared to bracket the concentration range of approximately 3.5 mg/mL (diluted with 0.9% Sodium Chloride Injection) and 35 mg/mL (undiluted). Tables 7 and 8 show that storing the diluted or neat formulation up to 5 hours at room temperature in dosing devices made from polyolefins.

Moreover, to provide flexibility of dose administration, another in-use study is performed to evaluate formulation (undiluted) compatibility with a syringe made from polypropylene (PP) and infusion line made from polyethylene (PE). No in-line filter is used. Table 9 shows that the prepared dosing formulation can be stored up to 4 hours at room temperature in dosing devices made from polyolefins.

TABLE 7
Simulated In-Use Results for 3.5 mg/mL Infusion Solution of
Formulation 2 Using PVC IV Bag and Infusion Line with PES.

| | | | | | Post- |
|---|---|---|---|---|---|
| | | | IV Bag | | Infusion |
| | | IV Bag | (2-hr hold | Pre-Infusion | (T = 3-hour |
| Assay | Units | (initial) | at RT) | (T = 0) | at RT) |
| Purity (SEC- | % | | | | |
| HPLC): | | | | | |
| Monomer | | 99.1 | 99.1 | 99.1 | 99.1 |
| Purity | | | | | |
| Total | | 0.8 | 0.8 | 0.9 | 0.8 |
| Aggregates | | | | | |
| pH | N/A | 6.1 | 6.0 | 6.0 | 6.0 |
| Appearance | N/A | Clear, | Clear, | Clear, | Clear, |
| | | colorless, no | colorless, no | colorless, no | colorless, no |
| | | visible | visible | visible | visible |
| | | particulates | particulates | particulates | particulates |
| HIAC | particles | | | | |
| ≥10 μm | per mL | 10≥ μm | 10≥ μm | 10≥ μm | 10≥ μm |
| | | 12/mL | 26/mL | 19/mL | 0/mL |
| ≥25 μm | | 25≥ μm | 25≥ μm | 25≥ μm | 25> μm |
| | | 0/mL | 0/mL | 7/mL | 0/mL |

TABLE 8
Simulated In-Use Results for 35 mg/mL Infusion Solution of
Formulation 2 Using PVC IV Bag and Infusion Line with PES.

| | | | | | Post- |
|---|---|---|---|---|---|
| | | | IV Bag | | Infusion |
| | | IV Bag | (2-hr Hold | Pre-Infusion | (T = 3-hour |
| Assay | Units | (Initial) | at RT) | (T = 0) | at RT) |
| Purity (SEC- | % | | | | |
| HPLC): | | | | | |
| Monomer | | 99.1 | 99.1 | 99.1 | 99.1 |
| Purity | | | | | |
| Total | | 0.8 | 0.9 | 0.8 | 0.9 |
| Aggregates | | | | | |
| pH | N/A | 6.1 | 6.1 | 6.1 | 6.0 |
| Appearance | N/A | Clear, | Clear, | Clear, pale | Clear, pale |
| | | colorless, no | colorless, no | yellow, no | yellow, no |
| | | visible | visible | visible | visible |
| | | particulates | particulates | particulates | particulates |
| HIAC | particles | | | | |
| ≥10 μm | per mL | 10≥ μm | 10≥ μm | 10≥ μm | 10≥ μm |
| | | 39/mL | 18/mL | 8/mL | 6/mL |
| ≥25 μm | | 25≥ μm | 25≥ μm | 25≥ μm | 25≥ μm |
| | | 0/mL | 0/mL | 0/mL | 1/mL |

TABLE 9
Simulated In-Use Results for 35 mg/mL Infusion Solution
of Formulation 2 Using PP Syringe and PE Infusion Line.

| | | | Post-Infusion | | |
|---|---|---|---|---|---|
| | | PP Syringe | T = 0.5 hr | T = 1 hr | T = 4 hr |
| Assay | Units | Initial | at RT | at RT | at RT |
| Purity (SEC- | % | | | | |
| HPLC): | | | | | |
| Monomer | | 99.2 | 99.2 | 99.2 | 99.2 |
| Purity | | | | | |
| Total | | 0.8 | 0.8 | 0.8 | 0.9 |
| Aggregates | | | | | |
| pH | N/A | 5.9 | 6.0 | 6.0 | 6.0 |
| Appearance | N/A | Clear, | Clear, | Clear, | Clear, |
| | | colorless, no | colorless, no | colorless, no | colorless, no |
| | | visible | visible | visible | visible |
| | | particulates | particulates | particulates | particulates |
| HIAC | particles | | | | |
| ≥10 μm | per mL | 10≥ μm | 10> μm | 10≥ μm | 10≥ μm |
| | | 52/mL | 39/mL | 25/mL | 7/mL |
| ≥25 μm | | 25≥ μm | 25≥ μm | 25≥ μm | 25≥ μm |
| | | 0/mL | 0/mL | 0/mL | 1/mL |

TABLE 10
Exemplary Drug Product Unit Formulas.

| | Quantity | Quantity | Quantity |
|---|---|---|---|
| | per Vial | per mL | per mL |
| Ingredient | (mg) | (mg) | (mg) |
| 2019-nCoV Ab | 35 | 150 | 40 |
| L-histidine | 1.55 | 1.55 | 1.55 |
| L-histidine | 2.10 | 2.10 | 2.10 |
| hydrochloride | | | |
| monohydrate | | | |
| mannitol | — | — | 45 |
| sucrose | 80.4 | 80.4 | — |
| polysorbate 80 | 0.5 | 0.5 | 0.2 |
| Water for Injection | q.s. to 1.0 mL | q.s. to 1.0 mL | q.s. to 1.0 mL |
| HCl Solution | pH | pH | pH |
| | adjustment | adjustment | adjustment |

### SEQUENCE LISTING

The following nucleic and/or amino acid sequences are referred to in the disclosure above and are provided below for reference.

SEQ ID NO: 1-Heavy Chain Determining Region 1 (HCDR1)
AASGFTVSSNYMS
SEQ ID NO: 2-Heavy Chain Determining Region 2 (HCDR2)
VIYSGGSTF
SEQ ID NO: 3-Heavy Chain Determining Region 3 (HCDR3)
ARVLPMYGDYLDY
SEQ ID NO: 4-Light Chain Determining Region 1 (LCDR1)
RASQSISRYLN
SEQ ID NO: 5-Light Chain Determining Region 2 (LCDR2)
YAASSLQS
SEQ ID NO: 6-Light Chain Determining Region 3 (LCDR3)
QQSYSTPPEYT
SEQ ID NO: 7-Heavy Chain Variable Region (HCVR)
EVQLVESGGGLVQPGGSLRLSCAASGFTVSSNYMSWVRQAPGKGLEWVSVIYSG
GSTFYADSVKGRFTISRDNSMNTLFLQMNSLRAEDTAVYYCARVLPMYGDYLD
YWGQGTLVTVSS
SEQ ID NO: 8-Light Chain Variable Region (LCVR)
DIVMTQSPSSLSASVGDRVTITCRASQSISRYLNWYQQKPGKAPKLLIYAASSLQS
GVPSRFSGSGSGTDFTLTISSLQPEDFATYYCQQSYSTPPEYTFGQGTKLEIK
SEQ ID NO: 9-Heavy Chain (HC)
EVQLVESGGGLVQPGGSLRLSCAASGFTVSSNYMSWVRQAPGKGLEWVSVIYSG
GSTFYADSVKGRFTISRDNSMNTLFLQMNSLRAEDTAVYYCARVLPMYGDYLD
YWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKRV
EPKSCDKTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMHEALH
NHYTQKSLSLSPGK
SEQ ID NO: 10-Light Chain (LC)
DIVMTQSPSSLSASVGDRVTITCRASQSISRYLNWYQQKPGKAPKLLIYAASSLQS
GVPSRFSGSGSGTDFTLTISSLQPEDFATYYCQQSYSTPPEYTFGQGTKLEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 11-2019-nCoV spike (S) protein (GenBank Accession No:
YP_009724390.1)
MFVFLVLLPLVSSQCVNLTTRTQLPPAYTNSFTRGVYYPDKVFRSSVLHSTQDLF
LPFFSNVTWFHAIHVSGTNGTKRFDNPVLPFNDGVYFASTEKSNIIRGWIFGTTLD
SKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGFS
ALEPLVDLPIGINITRFQTLLALHRSYLTPGDSSSGWTAGAAAYYVGYLQPRTFLL
KYNENGTITDAVDCALDPLSETKCTLKSFTVEKGIYQTSNFRVQPTESIVRFPNITN
LCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSK
VGGNYNYLYRLFRKSNLKPFERDISTEIYQAGSTPCNGVEGENCYFPLQSYGFQPT
NGVGYQPYRVVVLSFELLHAPATVCGPKKSTNLVKNKCVNFNFNGLTGTGVLTE
SNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPI
GAGICASYQTQTNSPRRARSVASQSIIAYTMSLGAENSVAYSNNSIAIPTNFTISVT
TEILPVSMTKTSVDCTMYICGDSTECSNLLLQYGSFCTQLNRALTGIAVEQDKNT
QEVFAQVKQIYKTPPIKDFGGFNFSQILPDPSKPSKRSFIEDLLFNKVTLADAGFIK
QYGDCLGDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLSSTASAL
GKLQDVVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRN
FYEPQIITTDNTFVSGNCDVVIGIVNNTVYDPLQPELDSFKEELDKYFKNHTSPDV
DLGDISGINASVVNIQKEIDRLNEVAKNLNESLIDLQELGKYEQYIKWPWYIWLG
FIAGLIAIVMVTIMLCCMTSCCSCLKGCCSCGSCCKFDEDDSEPVLKGVKLHYT

1. A pharmaceutical formulation comprising:
a 2019 novel coronavirus (2019-nCOV) antibody at a concentration from about 30 mg/mL to about 150 mg/mL;
a histidine buffer at a concentration from about 5 mM to about 40 mM;
sucrose at a concentration from about 8.0% (w/v) to about 8.5% (w/v); and
a polysorbate 80 at a concentration from about 0.02% (w/v) to about 0.08% (w/v), wherein the pharmaceutical composition has a pH from about 5.5 to about 7.0.

2. The pharmaceutical formulation of claim 1, wherein the 2019-nCOV antibody is at a concentration from about 30 mg/mL to about 40 mg/mL.

3. The pharmaceutical formulation of claim 1, wherein the 2019-nCOV antibody is at a concentration of about 35 mg/mL.

4. The pharmaceutical formulation of claim 1, wherein the histidine buffer concentration is about 20 mM.

5. The pharmaceutical formulation of claim 1, wherein the pH is about 6.0.

6. The pharmaceutical formulation of claim 1, wherein sucrose is at a concentration of about 8.05% (w/v).

7. The pharmaceutical formulation of claim 1, wherein polysorbate 80 is at a concentration of about 0.05% (w/v).

8. The pharmaceutical formulation of claim 1, wherein the 2019-nCOV antibody comprises three heavy chain heavy chain complementarity-determining regions (HCDR) and three light chain complementarity-determining regions (LCDR), and wherein HCDR1 comprises SEQ ID NO:1, HCDR2 comprises SEQ ID NO:2, HCDR3 comprises SEQ ID NO:3, LCDR1 comprises SEQ ID NO:4, LCDR2 comprises SEQ ID NO:5 and LCDR3 comprises SEQ ID NO:6.

9. The pharmaceutical formulation of claim 8, wherein the 2019-nCOV antibody comprises a heavy chain variable region (HCVR) comprising SEQ ID NO:7.

10. The pharmaceutical formulation of claim 8, wherein the 2019-nCOV antibody comprises a light chain variable region comprising SEQ ID NO:8.

11. The pharmaceutical formulation of claim 8, wherein the 2019-nCOV antibody comprises a heavy chain comprising SEQ ID NO:9.

12. The pharmaceutical formulation of claim 8, wherein the 2019-nCOV antibody comprises a light chain comprising SEQ ID NO: 10.

13. The pharmaceutical formulation of claim 1 further comprising one or more additional 2019-nCOV antibodies.

14. A method of treating 2019 novel coronavirus (2019-nCOV) infection or COVID-19 comprising the step of:
administering to an individual having a 2019-nCoV infection an effective amount of the pharmaceutical formulation of claim 1.

15. A method of preventing 2019 novel coronavirus (2019-nCOV) infection or COVID-19 in an individual comprising the step of:
administering to an individual at-risk for exposure or exposed to 2019-nCOV an effective amount of the pharmaceutical formulation of claim 1.


SEQUENCE LISTING (11 sequences)

SEQ ID NO: 1 - protein, 13 aa, Artificial Sequence
  Synthetic Construct
AASGFTVSSNYMS

SEQ ID NO: 2 - protein, 9 aa, Artificial Sequence
  Synthetic Construct
VIYSGGSTF

SEQ ID NO: 3 - protein, 13 aa, Artificial Sequence
  Synthetic Construct
ARVLPMYGDYLDY

SEQ ID NO: 4 - protein, 11 aa, Artificial Sequence
  Synthetic Construct
RASQSISRYLN

SEQ ID NO: 5 - protein, 8 aa, Artificial Sequence
  Synthetic Construct
YAASSLQS

SEQ ID NO: 6 - protein, 11 aa, Artificial Sequence
  Synthetic Construct
QQSYSTPPEYT

SEQ ID NO: 7 - protein, 119 aa, Artificial Sequence
  Synthetic Construct
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSS

SEQ ID NO: 8 - protein, 109 aa, Artificial Sequence
  Synthetic Construct
DIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIK

SEQ ID NO: 9 - protein, 449 aa, Artificial Sequence
  Synthetic Construct
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 216 aa, Artificial Sequence
  Synthetic Construct
DIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT